(19) United States
(12) Patent Application Publication
(10) Pub. No.: US 2010/0297742 A1
(43) Pub. Date: Nov. 25, 2010

(54) METHOD AND DEVICE FOR THERMAL ENZYMATIC HYDROLYSIS OF LIGNO CELLULOSE

(75) Inventors: Odd Egil Solheim, Hvalstad (NO); Hans Rasmus Holte, Reistad (NO)

Correspondence Address: BROOKS KUSHMAN P.C., 1000 TOWN CENTER, TWENTY-SECOND FLOOR, SOUTHFIELD, MI, 48075, US

(73) Assignee: CAMBI AS, Asker (NO)

(21) Appl. No.: 12/438,426

(22) PCT Filed: Aug. 23, 2007
(86) PCT No.: PCT/NO07/00298

§ 371 Date: Feb. 23, 2009

(30) Foreign Application Priority Data
Aug. 30, 2006 (NO) 20063872

(51) Int. Cl. C12P 19/00

(52) U.S. Cl. 435/277

(57) ABSTRACT

A method for thermal enzymatic hydrolysis of ligno cellulose is provided by feeding a raw material based on ligno cellulose, pH-adjusting and other additives and possibly thinner liquid into one or more reactors, supplying steam to heat and increase the pressure in the reactor, relieving the pressure in the reactor in two steps and provide a hydrolysing of the treated material based on ligno cellulose, mixing the treated material with an enzyme solution and thinner fluid in a mixing step, and feeding the mixture to a liquefaction step and enzymatic saccharificate the treated ligno cellulose material.

The present invention relates to a method by thermal enzymatic hydrolysis of raw materials that contain ligno cellulose according to the ingress of claim 1.

Climate researchers have in the last decades observed rapid climate changes and an increase of the temperature in the atmosphere. These changes have been coupled to an increase of CO₂ in the atmosphere, caused by the increased consumption of fossil fuel. CO₂ is an important contributor to the greenhouse effect. Because of this, there is an increasing interest in finding renewable energy sources that can reduce the fossil energy consumption in the world.

As a consequence of the industrial and economic development in the world, we have a situation where the energy consumption of the world has increased heavily over a very long period of time. As a result of the lack of stable energy production to cover the increased consumption, combined with that it does not seem to be any clear energy sources that can take over the role of the old energy sources, a situation with lack of energy has aroused. The result is that stable high energy prices have been established. This situation with high energy prices has now made it attractive to develop new methods for production and refining of renewable energy.

As a consequence of these two facts, 1) climate changes, and 2) lack of energy/high prices, there is now focus on renewable energy resources.

Ethanol is an energy carrier that can be mixed into petrol, and in that way utilize existing distribution systems and means of transportation, actually over the whole world. Ethanol can be produced from renewable raw materials.

Another advantage is that countries without their own oil, gas or coal resources can now use available areas to produce raw materials for ethanol fermentation, and in this way substantially improve the trading balance.

Another advantage is that countries without their own oil, gas or coal resources can now use available areas to produce raw materials for ethanol fermentation, and in this way substantially improve the trading balance.

Today, ethanol is produced from three different groups of raw materials;

1) Sugar producing plants where the sugar can be fermented directly to ethanol. Examples of such plants are sugar turnips and sugar beets. Especially around the equator, the production of sugar turnips forms the basis for a substantial ethanol industry.
2) Amyl based raw materials. Examples of amyl based raw materials are grain, corn and potatoes. At first, amyl must be converted to sugar by means of enzymes before fermentation to ethanol. There is now a rapid increasing ethanol production based on grain and corn in North America and Europe. This growth in using grain and corn for ethanol production has already lead to price increase on these raw materials, which influences the profitability. In a longer perspective, the shortage of amyl based raw materials will lead to an additional pressure on the price, which will make it more attractive to produce energy raw material instead of food to the increasing population of the world. Such a situation would at a time become a limitation for further growth within the use of amyl based raw materials for sugar production.
3) Raw materials based on ligno cellulose consist mainly of cellulose, hemi cellulose and lignin. In addition, there are generally resin and smaller amounts of other compounds. Ligno cellulose opens up a totally new potential for raw material of a large extent for ethanol production. Cellulose and hemi cellulose must at first be converted to sugar before it can be fermented. From the nature's side, cellulose is usually impregnated with lignin, resin and other binding materials, and an industrial method requires powerful pre-treatment to make the cellulose bindings available for conversion to sugar. Conversion of cellulose to sugar can be done chemically or enzymatically. Ethanol from ligno cellulose has so far not been established as a commercial large marked, except as a bi product of existing cellulose factories. In the later years, large resources have been brought into action to develop techniques to make this raw material available for ethanol production. Examples of such raw materials are straws, bran, rests of corn plants, paper, mud, organic waste, splinter, etc.

Methods for thermal hydrolysis have been developed for treatment of different types of organic material. Examples of this are thermal hydrolysis of mud and food waste before anaerobic digestion for production of bio gas. Bio gas contains methane, which among others can be used as fuel in conventional gas engines, turbines, boilers and for drying processes. Mud and food waste treated this way has usually a content of dry substances of <20%.

Methods for thermal hydrolysis have also been developed for raw materials with higher content of dry substances, to for instance production of fuel pellet from splinters and shavings. In such plants it is in addition, dependent on the raw material, necessary to remove water at drying before and/possibly after the thermal hydrolysis to produce dry and stable fuel pellet. Other ranges of application for thermal hydrolysis have been production of the sweetener substance xylitol, a sugar substance constituted by hemi cellulose, and for production of animal food.

From U.S. Pat. No. 4,321,328, a process for production of ethanol and fuel products is known. The process has a mixing step for cellulose solid state with water so that the saccharification tank is added a uniform solution with the correct viscosity and temperature. A mixture containing ethanol is used as a recycling diluting liquid at the front edge of an enzymatic hydrolysis in the saccharification tank. In the mixing chamber, the solid substance is mixed with recycled substance from the fermenting tank.

From WO 2006/032282, it is known a method for mixing bio mass or organic waste containing ligno cellulose, so that the sugar is made more available for further enzymatic hydrolysis and fermentation. Thermal hydrolysis, oxidation and steam explosion are used.

From US 2004/0016525 it is also known to use steam explosion in connection with treatment of ligno cellulose.

From WO 2006/024242 it is known a complex system for continuous pressure hydrolysis of ligno cellulose substance and amyl, followed by a two step phase of expansion and separation of a gas phase and a hydrolyzat solution. Amyl substance is added to soluble glucose and sugar from the hydrolysis, and the treatment is treated with amolytic enzymes.

A large problem with prior known solutions for production of ethanol from cellulose and hemi cellulose is that thin solutions are used, i.e. a low content of dry substance. This result in that thin sugar solutions are formed, which again gives a low concentration of ethanol after fermentation. This imply that such plants becomes unreasonable large and brings along a high energy consumption.

The purpose of the present invention is therefore to be able to treat solutions with a high content of dry substance, and thereby achieve a sugar concentration in the order of 7-10% This will result in that the size of the plant can be reduced and at the same time achieve a considerable reduction of the energy consumption.

This invention comes from the above mentioned experiences, but used as a process for pre-treatment of raw materials based on lingo cellulose to fermentation of ethanol. Substantial new developments have been done through research and development to establish a technical economic viable process.

The process is suitable for utilization of enzymes to convert cellulose to C6-sugar and hemi cellulose to C5-sugar. The purpose of thermal hydrolysis is to soften lignin and other binding materials to weaken the bindings and to make cellulose and hemi cellulose available so that enzymes can produce C6- and C5-sugar. C5- and C6-sugar can then be converted to ethanol through fermentation after the process by using anaerobe bacterial systems. Today, several enzyme producers are purposeful working with the development of enzymes for conversion of cellulose/hemi cellulose.

Many research communities are nowadays working with the development of cultures of bacteria that can converted both C5- and C6-suger to ethanol. It is of large commercial interests to develop robust and highly effective cultures with such qualities, and the dominant cultures that fulfil such requirements seem to be thermopile.

The thermal enzymatic hydrolysis process that is developed here for pre-treatment of substances containing lingo cellulose are very flexible regarding type of raw material and content of dry substance. The process is characterized by the energy economic important heat recycling and that the substance is handled as solid substance until it is continuously fed into the liquefaction step, where conversion of cellulose to C6-sugar and hemi cellulose to C5-sugar takes place. This conversion is called saccharification. Under the saccharification, cellulose and hemi cellulose fibres in solid form are split in C6- and C5-sugar in fluid form. This leads to that the viscosity of the mixtures is changed and that the mixture can be handled as a thick liquid in the so called liquefaction tank. Fluid sugar solution (C5 and C6) are gradually drained off the tank while new solid substance is dosed into the tank.

Handling of solid substance and the handling of fluid substances set different requirements to process equipment. The intermediate phase between solid and fluid can be difficult to handle. It is therefore important to divide between these phases. The present invention does precisely this through a controlled transition from solid substance to fluid phase by that a small amount of solid substance is fed into a volume where the saccharification is going on and a substantial change in viscosity has made the mixture fluid. The dosing of solid substance is controlled by the speed of saccharification and the viscosity of the fluid mixture.

The liquefaction tank is a well stirred tank, where thermal hydrolyzed substance is continuously fed into a mixing point in a pump circulation circuit of the tank. The pump circulation circuit also functions as a cooling circuit with a heat exchanger. An effect of the thermal hydrolysis is that the raw material is sterilized, and it is important that the material is kept sterile to avoid contamination of the saccharification process and the following (downstream) fermentation process.

Enzymes are added and well mixed in the liquefaction tank.

Oxidants, such as i.e. oxygen or hydrogen peroxide, can be added before the liquefaction step to increase the efficiency of the thermal hydrolysis treatment through delignification and a starting depolymerisation of cellulose and hemi cellulose. Oxygen will lead to a decomposition of lignin (delignification) to organic acids. Oxygen will also start a breakdown (depolymerisation) of cellulose and hemi cellulose, and like this contribute to a saccharification of C5- and C6-sugar. However, exaggerated use of oxygen will also lead to breakdown of C5- and C6-sugar which gives mass loss and reduce sugar profit. The oxidation gases must be evacuated through pressure decompression in the top of the reactor if it, on the other hand, is desirable with a mass reduction, which can be interesting for certain types of raw material, to reduce the amount of rest substance and the wish is to use the heat and pressure generated from the oxidation.

Other additives can also be added and organic acids recycled to adjust the pH and achieve as much optimal profit as possible of the process. Pre-heated process liquid from the heat recycling tank can be used as thinning liquid to the raw material to achieve an advantageous content of humidity for the process.

Pre-heated process liquid from the heat recycling tank can also be used as thinner liquid in the liquefaction tank to achieve an acceptable viscosity for optimal stirring of the tank and pumping of the mixture.

Pre-heated process liquid from the heat recycling tank is rich on organic acids, and it can be used as heat and thinner liquid in downstream fermentation process. Excess liquid rich on organic acids can be used as food for anaerobic digestion to produce bio gas.

The objective of the present invention is to exploit raw material that contains ligno cellulose for production of alcohol. The method converts cellulose to C6-sugar and hemi cellulose to C5-sugar by a thermal enzymatic hydrolysis. Heat recycling does the process energy economic profitable. Development of commercial processes for production of ethanol from raw materials that contain ligno cellulose will open up a new and broader spectre of raw materials for production of ethanol.

These and other objects are achieved by a method for thermal enzymatic hydrolysis of ligno cellulose, which is characterized in that the method comprises the following steps:
lead a raw material based on ligno cellulose, pH-adjusting and other additives and possibly thinner liquid into one or more reactors,
supply steam to heat and increase the pressure in the reactor,
relieve the pressure in the reactor in two steps and provide a hydrolysation of the treated material based on ligno cellulose,
mix the treated material with an enzyme solution, and sugar solution from a following liquefaction step and thinner fluid in a mixing step, where the amount of dry substance in the mixture after the mixing step is in the order of 20-40%,
to lead the mixture to a liquefaction step and enzymatic saccharificate the treated ligno cellulose material.

The method comprises preferably at least two reactors, which reactors are operated sequential.

The temperature in the reactor is preferably in the order of 150° C. to 225° C. (corresponding to 5-25 bar), preferably in the order of 160° C. to 207° C. (corresponding to 6-18 bar) and the retention period in the reactor is preferably in the order of 2 to 15 minutes.

The mixing step comprises preferably a feed screw which feeds solid substance axial towards a rotating mixing and milling unit, which feed screw ends in a concentric pipe with the equivalent dimension as the feed screw, with an external pipe segment with a larger dimension where liquid from the liquefaction step is fed and where liquid from the external pipe is mixed with solid substance from the inner pipe into the inlet side of the mixing and milling unit, after which mixed material from the outlet side of the mixing and milling unit is led to the liquefaction step.

The invention will be explained in detail in the following by means of an example embodiment, with reference to the appended drawing.

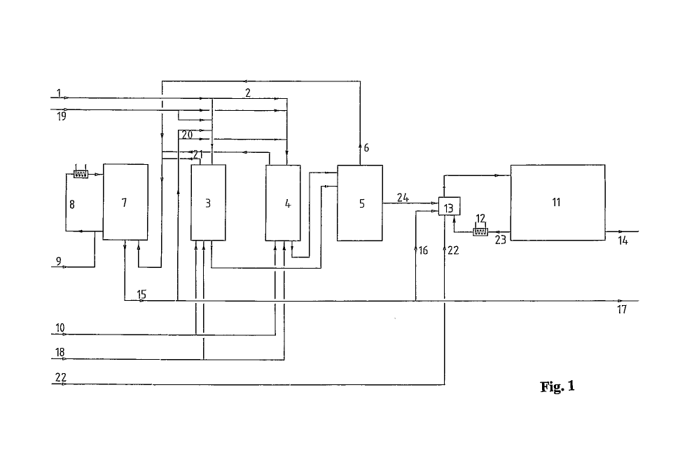

FIG. 1 is a block diagram which schematic shows the different steps in the method according to the invention.

At first, a raw material based on ligno cellulose (1) is fed by means of a dosing system (2) into one or more reactors (3 and 4). Additives (19) can be added to adjust the pH and increase the profit of the process. Pre-heated thinner liquid (20) can also be added to soften the raw material and to recycle volatile organic acids from the heat recycle tank (7), and to adjust the pH as well as to achieve an improved packing of the raw material in the reactors (3 and 4). Organic acids contribute to lower the pH in the mixture, and thereby weaken the bindings in the ligno cellulose material and thereby contribute to a more efficient process.

If more than one reactor is used, the reactors (3 and 4) will work sequentially. When the first reactor (3) has been filled with raw material, the material is pre-heated with steam (10) from an external source or with flash steam (21) from the other reactor (4). After pre-heating, the reactors become heated with steam (10) added in the bottom of the reactor to achieve pressure and temperature between 150° C. and 225° C. (corresponding to 5-25 bar), preferably between 160° C. and 207° C. (corresponding to 6-18 bar). If oxygen is added as additive to increase the profit, the oxygen will also contribute to an increase of temperature and pressure corresponding to the added amount of oxygen that will oxidize. Without oxidizing additives, added steam will alone stand for the increase of desired pressure and temperature in the reactor.

After a desired retention period in the reactor in the range of 2-15 minutes, the pressure in the reactor (3) is relieved. The pressure can be relieved to the heat recycle tank (7) or transferred to the other reactor (4) which now in advance has been filled with a new raw material. The rest pressure in the reactor (3) is used to transport the material from reactor (3) to the flash tank (5). The rest pressure in the reactor will typically be 5-15 bar, preferably 10-12 bar, depending on the nature of the raw material.

As a consequence of the large pressure difference between the reactor and the flash tank, the steam explosion that then happens will lead to that the material is torn apart from each other. Lignin, resin and other binding materials are softened or melted in the reactor, and cellulose and hemi cellulose are freed after the steam explosion. Cellulose and hemi cellulose will then have been made available for enzymatic hydrolysis. Further optimization of pressure, temperature and retention period will be done for each type raw material containing ligno cellulose. The same is valid for the amount of additives and recycling as a result of the different characteristics and composition of the raw materials.

Reactor (4) is heated with steam (10) in the same way as reactor (3) when flash steam from reactor (3) is recycled to reactor (4). Flash steam from the reactor (4) is recycled back to reactor (3) in the same way after reactor (3) is filled with new raw material. For larger plants, to or more rectors will be implemented with a common heat recycle system (21) between the reactors. One to six reactors would normally have common heat recycle tank (7) and flash tank (5). The requirement for redundancy in the plant is vital for the number of reactors per line.

As a basis, the flash tank (5) is atmospherically, but under emptying from the reactor it will be a small over pressure. Steam is flashed off and separated in the flash tank (5). Flash steam (6) is drained off and retrieved in a heat recycle tank (7), where the steam condensates in contact with cold liquid. In addition to steam, the flash steam contains volatile compounds, something which leads to a concentrated content of organic acids in the heat recycle tank (7). Typical pH in the heat recycle tank (7) will be in the area of 3-7, depending on the degree of thinning. The thinner water (9) is added to the heat recycle tank to condensate flash steam (6) and to add sufficient thinner liquid (15) to the process. To limit the amount of thinner water (9) the heat recycle tank (7) can be equipped with a cooler circuit (8).

The bottom of the flash tank (5) is equipped with a plug mate system (24) to handle solid substance and to prevent re-feeding of liquid from the liquefaction step (11). The thermal hydrolysed material from the flash tank is fed as a plug, for instance with a screw feed, into the liquefaction step (11). The injection point should preferably be a suitable mixing point (13) outside the liquefaction tank itself, with a quick mixing of solid material into the fluid phase from the liquefaction tank (11). A preferable device for mixing outside the tank is a screw feed which feeds solid substance (24) axially towards a rotating mixing and milling unit. The screw feed ends in a concentric pipe of corresponding dimension as the screw feed with an external pipe piece of a larger dimension where liquid (23) from the liquefaction tank (11) is added. Liquid from the external pipe is mixed with the solid substance from the inner pipe in on the inlet side of the mixing and milling unit. Well mixed material from the outlet side of the mixing and milling unit is then fed back to the liquefaction tank (11).

To achieve an acceptable viscosity for a satisfactory stirring and pump characteristics, one can, depending on the characteristics of the raw material, add thinner liquid (16) in relation to the mixing point (13). Acceptable content of solid material is in the order of 20-40%, but the character of the raw material will be vital for how high content of solid material will be possible. The solid material in the flash tank is hot, and the temperature must be reduced for enzymatic hydrolysis. Cooling is done in fluid phase on the circulation circuit on the liquefaction tank (12), as heat transfer will be better in fluid phase than in solid substance. A flash tank can feed one or more liquefaction tanks in parallel.

Enzymatic hydrolysis should preferably be preformed in the temperature area between 50° C. and 90° C. New mixes of enzymes are constantly being developed, and the present invention is especially suitable for thermophilic enzyme mixes. By correct and stable temperature the enzymes (22) are mixed into the liquefaction step, preferably at the inlet of the mixing device (13). After saccharification to C6- and C5-sugar, the mixture is fed to downstream process for fermentation to alcohol as a product stream (14).

As a result of this invention a number of raw materials based on ligno cellulose will be made available for technical and economical profitable production of ethanol.

With the present invention, an ethanol factory producing 100 000 m³ ethanol depending on the composition of the raw material will consume 300 000-500 000 tons solid substance of raw materials based on ligno cellulose.

### LIST OF REFERENCE NUMBERS

1 Raw material (ligno cellulose)
2 Dosing system (not shown in detail)
3 Reactor
4 Reactor
5 Flash tank
6 Flash steam
7 Heat recycle tank
8 Cooling circuit
9 Thinner water
10 Steam
11 Liquefaction step
12 Heat exchanger
13 Mixing device
14 Product solution containing sugar
15 Thinner liquid
16 Thinner liquid
17 Excess liquid from heat recycle tank
18 Oxidation means
19 Additives
20 Thinner fluid
21 Stress relieving steam
22 Enzyme solution
23 Liquid from the liquefaction step
24 Thermal hydrolysed mass from the flash tank

1. Method for thermal enzymatic hydrolysis of ligno cellulose, comprising the following steps:
lead a raw material based on ligno cellulose, pH-adjusting and other additives and possibly thinner liquid into one or more reactors,
supply steam to heat and increase the pressure in the reactor,
to relieve the pressure in the reactor in two steps and provide a hydrolysing of the treated material based on ligno cellulose,
characterized by the additional steps:
mix the treated material with an enzyme solution, and sugar solution from a following liquefaction step and thinner fluid in a mixing step, where the amount of dry substance in the mixture after the mixing step is in the order of 20-40%,
lead the mixture to a liquefaction step and enzymatic saccharificate the treated ligno cellulose material,
recover volatile organic acids and the heat from the flash steam to be used in a combined thermal and dilute acid hydrolysis by diluting materials in the steam reactors,
said mixing step comprises a screw feed which feeds solid substance axially towards a rotating mixing and milling unit, which screw feed ends in a concentric pipe with corresponding dimension to the screw feed, with an external pipe piece with a larger dimension where liquid from the liquefaction step is added and where liquid from the outer pipe is mixed with solid substance from the inner pipe into the inlet side of the mixing and milling unit, after which mixed material from the outlet side of the mixing and milling unit is led to the liquefaction step.

2. Method according to claim 1, characterized in that it comprises at least two reactors, which reactors operates sequentially.

3. Method according to claim 1, characterized in that the temperature in the reactor is in the magnitude of 150° C. to 225° C. (corresponding to 5-25 bar), preferably in the magnitude 160° C. to 207° C. (corresponding to 6-18 bar) and that the retention period in the reactor is in the magnitude 2 to 15 minutes.

4. Method according to claim 2, characterized in that the temperature in the reactor is in the magnitude of 150° C. to 225° C. (corresponding to 5-25 bar), preferably in the magnitude 160° C. to 207° C. (corresponding to 6-18 bar) and that the retention period in the reactor is in the magnitude 2 to 15 minutes.
